(19) United States
(12) Patent Application Publication
(10) Pub. No.: US 2016/0073854 A1
(43) Pub. Date: Mar. 17, 2016

(54) SYSTEMS AND METHODS USING SPATIAL SENSOR DATA IN FULL-FIELD THREE-DIMENSIONAL SURFACE MEASUREMENT

(71) Applicant: APERTURE DIAGNOSTICS LTD., Cutchogue, NY (US)

(72) Inventor: Robert ZEIEN, Cutchogue, NY (US)

(73) Assignee: APERTURE DIAGNOSTICS LTD., Cutchogue, NY (US)

(21) Appl. No.: 14/823,301

(22) Filed: Aug. 11, 2015

Related U.S. Application Data
(60) Provisional application No. 62/049,628, filed on Sep. 12, 2014.

(51) Int. Cl. A61B 1/00; A61B 1/06; A61B 1/04

(52) U.S. Cl. CPC A61B 1/00009; A61B 1/041; A61B 1/0661; A61B 1/00016; A61B 1/00006

(57) ABSTRACT

Embodiments of the present disclosure may utilize one or more spatial sensors, such as an accelerometer, gyroscope, and/or IMU, in providing position and/or orientation data for continuous real-time, full-field, and three-dimensional (“3-D”) surface data maps. An electromagnetic radiation source is configured to project electromagnetic radiation onto a surface. An image sensor is configured to capture image data representing the projected pattern as reflected from the surface. One or more spatial sensors are used to provide coordinate data for providing the position and/or orientation information. Using the coordinate information, an image processing module may be configured to stitch together the individual full-field 3-D data frames into a complete representation of the surface under study.

### RELATED APPLICATIONS

The present application claims priority to U.S. Provisional Application Ser. No. 62/049,628 filed Sep. 12, 2014, the disclosure of which is incorporated herein by reference in its entirety.

This application is also related to U.S. patent application Ser. No. 13/830,477, entitled “Full Field Three-Dimensional Surface Measurement”, filed on Mar. 14, 2013, which is also incorporated herein by reference in its entirety.

### FIELD OF INVENTION

The present invention generally relates to systems and methods for three-dimensional surface measurement.

### BACKGROUND

As discussed in U.S. patent application Ser. No. 13/830,477, accurate three-dimensional maps of external and internal human body surfaces are necessary for many medical procedures. Current techniques for three-dimensional scanning of external and internal body surfaces have many drawbacks. For example, laser-based scanning typically requires a patient to remain motionless, with even minor movements affecting the accuracy of the scan. A typical laser scan may require a patient to sit still for ten to fifteen seconds while many two-dimensional slices are gathered. The two-dimensional slices are later recompiled into a three-dimensional representation of a surface. Movement during this time period by the patient, including respiration, tremors, or muscle reflexes, can negatively impact the accuracy of the scan.

Scanning techniques can result in inaccurate mapping if there are inaccuracies in the data regarding the positioning and/or orientation of the scanning equipment. A need exists for three-dimensional surface measurement techniques that can be performed quickly and accurately.

### BRIEF DESCRIPTION OF THE DRAWINGS

FIG. 1 illustrates a system for obtaining position and/or orientation data according to-example embodiment of the present invention.

FIG. 2 illustrates a logic flow according to an example embodiment of the present invention.

FIGS. 3A-B illustrate representative views of an endoscope according to an example embodiment of the present invention.

FIG. 4 illustrates a distal end according to an example embodiment of the present invention.

FIG. 5 illustrates representative views of a capsule according to an example embodiment of the present invention.

### DETAILED DESCRIPTION

Reference will now be made in detail to embodiments, examples of which are illustrated in the accompanying drawings. In the following detailed description, numerous specific details are set forth in order to provide a thorough understanding of the present invention. However, it will be apparent to one of ordinary skill in the art that the present invention may be practiced without these specific details. In other instances, well-known methods, procedures, components, and circuits have not been described in detail so as not to unnecessarily obscure aspects of the embodiments. Wherever possible, like reference numbers are used for like elements.

Embodiments of the present invention relate to systems incorporating components for obtaining position and/or orientation data of a measurement package (or an image sensor or another suitable reference) in a system for full-field, three-dimensional (“3-D”) surface mapping, and related methods. The system for full-field, 3-D mapping may be similar to those disclosed in U.S. patent application Ser. No. 13/830,477, used to perform measurement of surfaces, such as external and internal surfaces of the human body, in full-field and in 3-D. Full-field may refer to the ability of a device's sensor to capture and compute 3-D information of an entire scene containing an object being measured, for example. Real-time may refer to use of sufficiently fast sensor exposures or frame-rates to minimize or eliminate perceptible target surface motion, for example.

As disclosed in U.S. patent application Ser. No. 13/830,477, the system may include an electromagnetic radiation source, which may be configured to project electromagnetic radiation onto a surface. The electromagnetic radiation source may be configured to project the electromagnetic radiation in a pattern corresponding to a spatial signal modulation algorithm. The electromagnetic radiation source may also be configured to project the electromagnetic radiation at a frequency suitable for transmission through the media in which the radiation is projected. An image sensor may be configured to capture image data representing the projected pattern that is reflected, i.e., the reflected spatially modified signal pattern. An image-processing module may be configured to receive the captured image data from the image sensor and to calculate a full-field, 3-D representation of the surface using the captured image data and the spatial signal modulation algorithm. A display device may be configured to display the full-field, 3-D representation of the surface.

Embodiments of the present invention may be further integrated into a probe, diagnostic or therapeutic catheter, endoscope, or a capsule to allow full-field, 3-D surface replication of internal surfaces of the human body. Such a device may be internally or externally guided, steerable or propelled in order to be advanced to, or navigated through cavities or the cardiovascular system.

FIG. 1 illustrates a real-time, full-field, 3-D surface replication system 100 with one or more components for obtaining position and/or orientation data of a measurement package (or image sensor or other suitable reference) according to an example embodiment of the present invention.

As shown in FIG. 1, system 100 may include a measurement package 102, a controller system 106, and a display system 108. System 100 may implement the spatial signal modulation (SSM) techniques described in U.S. Pat. No. 5,581,352 filed on Feb. 27, 1995, the entirety of which is hereby incorporated by reference, to reproduce instant, quantifiable 3-D maps of external and internal surfaces of the human body.

Measurement package 102 may include an image sensor such as a camera device 110 and a radiation source 112. The radiation source 112 may be fabricated by placing a slide or grating (not shown) with a desired pattern between a radiation emitting device and a lens (not shown). The camera device 110 may be a device capable of capturing image data reflected from the target surface 104 (e.g., a charge-coupled device (CCD) camera).

One or more spatial sensor(s) 114 are used to obtain position and/or orientation data of the measurement package 112. The spatial sensor(s) 114 can obtain data regarding the position and/or orientation of the sensor(s) 114 or of another suitable reference point that can be used in determining the position and/or orientation from which the image is being taken (e.g., the camera device 110). The spatial sensor(s) 114 providing data regarding position and/or orientation may be adapted to provide coordinate information along six axes (i.e., x, y, z, roll, pitch, and yaw). Examples of spatial sensor(s) 114 include accelerometers and gyroscopes, which provide data that can be used to determine position and/or orientation. For example, the sensor(s) 114 may be an inertial measurement unit (IMU) as is known in the art, capable of providing position and orientation information. The sensor(s) 114 may provide position and/or orientation information by detecting forces and deviations in their intensity. Thus, the sensor(s) 114 may be used to detect changes in a movement direction as well as the degree of displacement of the measurement package 102.

In some instances, the spatial sensor(s) 114 may be implemented using micro-electromechanical systems (MEMS) accelerometers and/or gyroscopes. For example, spatial sensor(s) 114 may be implemented on one or more MEMS integrated chips. MEMS components can be generally between 1 and 100 micrometers in size (i.e., 0.001 to 0.1 mm). Thus, MEMS accelerometers and/or gyroscopes may provide accurate navigation and orientation information for even the smallest 3-D optical measurement package 102.

Controller system 106 (or image processing module) may include a processor or state machine capable of receiving image data captured by the camera device 110, receiving data captured by the spatial sensor(s) 114, and processing the data to calculate a full-field, 3-D representation of the target surface 104. Using the position/orientation data obtained from the spatial sensor(s) 114, the controller system 106 may similarly reconstruct the position/orientation of the target surface 104. Such a machine may include, for example, any suitable processing platform, computing platform, computing device, processing device, computing system, processing system, computer, processor, or the like, and may be implemented using any suitable combination of hardware and/or software.

The data from the spatial sensor(s) 114 is used to determine the position and/or orientation of the measurement package 102 (or the image sensor or a similar suitable reference) at the time of the taking of the captured image data. The controller system 106 (or image processing module) uses this position and/or orientation data in order to properly determine the relative positioning of the captured image data in calculating the full-field, 3-D representation of the target surface 104.

Display system 108 may include a display device (liquid crystal display device, light emitting diode display device, etc.) to receive the full-field, 3-D representation of target surface 104 from the controller system 106 and display the digital representation of the surface 104 to be analyzed by a user.

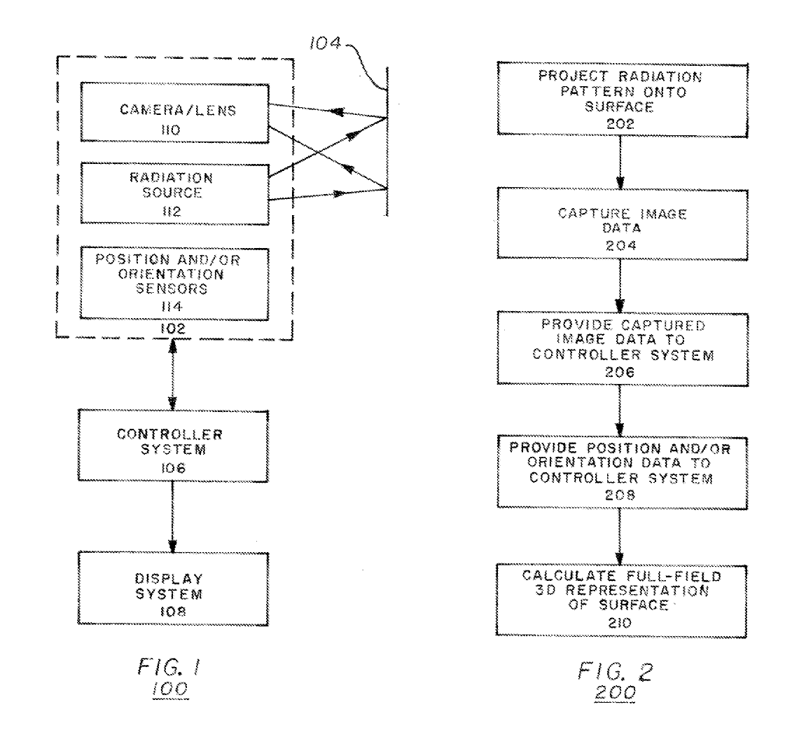

FIG. 2 is a logic flow 200 of an operation of the replication system 100 of FIG. 1 according to an example embodiment of the present invention.

During operation, radiation source 112 may project a pattern of electromagnetic radiation, according to a spatial signal modulation algorithm, onto a target surface 104 (step 202). The pattern may take the appearance of parallel bands of electromagnetic radiation, for example. According to embodiments of the present invention, the carrier frequency of the projected spatial radiation signals may depend on the media that the signals are propagating through. For example, human blood is some 2,500 times more transparent at certain infrared frequencies versus shorter wavelengths in the visible blue range. It is also not possible to use electromagnetic radiation to “see” an object if the wavelength of the radiation used is larger than the object. Thus, the emitter carrier frequency may be chosen based upon one or more characteristics (e.g., particle size, color, quantity of particles, etc.) of a media (e.g., air, blood, mucus, urine, etc.) adjacent to a target surface.

The spatial signals may reflect from the target surface 104 back to the camera device 110. The camera device 110 may capture the reflected spatial signals, which are changed/modulated by interaction with the surface 104 (step 204). The captured reflection images of the distorted projections contain spatially encoded 3-D surface information. Data representing the reflected (and distorted) spatial signals may be transmitted to the controller system 106 for processing (step 206).

The spatial sensor(s) 114 may gather coordinate information (e.g., position and/or orientation information) of measurement package 102 at the time of image data acquisition and that information may be provided to the controller system 106 (step 208). For example, a variety of coordinate information may be provided for along different axes (i.e., x, y, z, roll, pitch, and yaw). Using the coordinate information, position and orientation information may be calculated and utilized by the controller system 106 in the mapping, or stitching together, of individual full-field 3-D data frames into a complete representation of the surface under study. It will be understood that the order of steps need not be as shown in FIG. 2. For example, step 208 may take place before step 206.

Controller system 106 may include an image processing module and may use existing information to isolate the content of the reflected spatial signal that contains the 3-D shape information. The shape information may be used to mathematically reconstruct the 3-D shape of target surface 104 (step 210). The full-field surface map's spatial location and orientation may be generated using the coordinate information obtained from the spatial sensor(s). Controller system 106 may transmit digital data corresponding to the calculated representation of the surface 104 to the display system 108 to display a digital image representing a 3-D view of the surface 104.

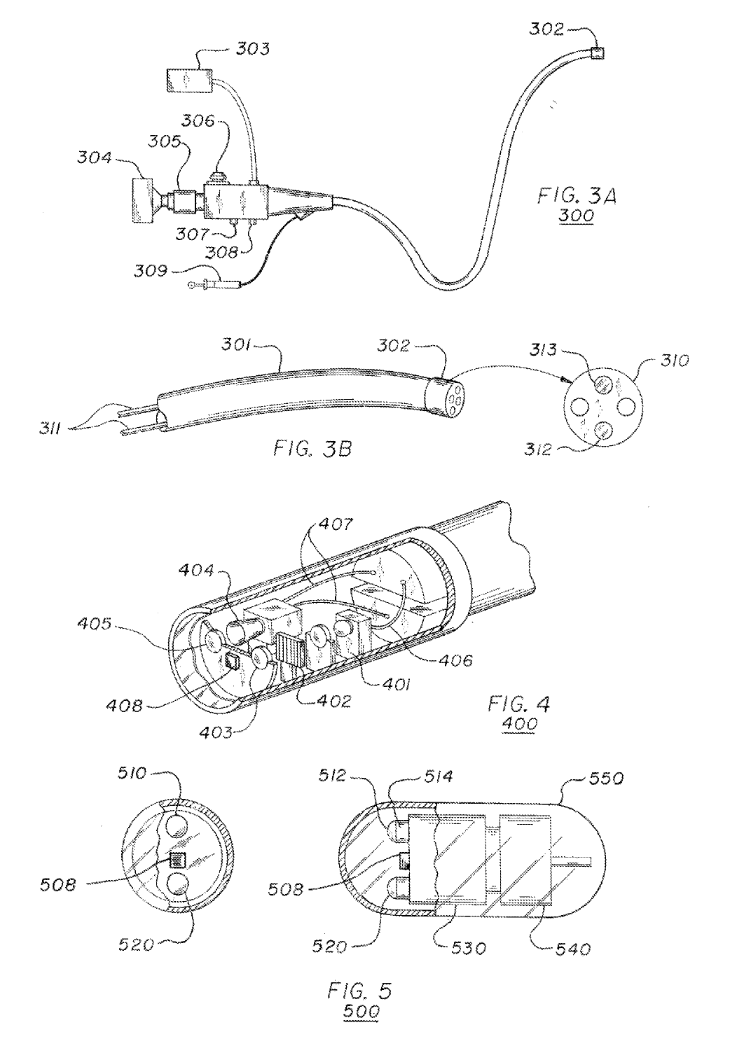

FIGS. 3A-B illustrate representative views of an endoscope according to example embodiments of the present invention.

As shown in FIG. 3A, endoscope 300 may be used to examine interiors of internal human organs/cavities and generate full-field, 3-D representations of the organs/cavities. Endoscope 300 may include a catheter section 301, a distal end 302, a camera 304 (similar to camera 110 of FIG. 1), and a radiation source 303 (similar to radiation source 112 of FIG. 1). The camera 304 and radiation source 303 may be connected to the catheter section 301 on one end of the catheter section 301 and the distal end 302 may be connected to the catheter section 301 on another end of the catheter section 301. In other embodiments, the camera 304 and radiation source 303 may both be located at the end of catheter section 301 opposite distal end 302, the camera 304 and radiation source 303 may both be located at the end of catheter section 301 at distal end 302, or the camera 304 and radiation source 303 may be located at opposite ends of catheter section 301.

Catheter section 301 may be a flexible shaft and may include a number of channels (not shown) which may facilitate an examination of a patient's body. The channels in the catheter section 301 may run from one end of the catheter 301 to another end to allow transmission of data between camera 304, radiation source 303 and distal end 302 (described in further detail below). The channels may permit a physician to engage in remote procedures such as transmission of images captured by the distal end 302, providing radiation generated by the radiation source 303 to distal end 302, irrigation for washing and removing debris from distal end 302 (e.g., using air/water pathway 307 and suction pathway 308), and introduction of medical instruments into a patient (e.g., via instrument pathway 309).

Operation of an endoscope according to an embodiment of the present invention will now be described with respect to FIGS. 3A and 3B. FIG. 3B illustrates a detailed view of catheter section 301 of endoscope 300 according to an embodiment of the present invention. Catheter section 301 may include distal end 302 and a fiber optics bundle 311.

Distal end 302 may include a distal tip 310 with projection optics 312 and imaging optics 313. The projections optics 312 and imaging optics 313 may each include a lens to focus the radiation used by the endoscope 300. Lenses may be used to focus radiation, and may include optical lenses, parabolic reflectors, or antennas, for example. Fiber optics bundle 311 may connect radiation source 303 to projection optics 312 to facilitate transmission of electromagnetic radiation from radiation source 303 to projection optics 312. Fiber optics bundle 311 may also connect camera 304 to imaging optics 313 to facilitate transmission of imaging data captured by imaging optics 313 to camera 304.

During a medical procedure, distal end 302 and catheter shaft 301 may be inserted into a patient and guided to a surface inside the patient's body that is under examination. Once the distal end 302 is properly oriented, the radiation source 303 may transmit a spatial pattern of electromagnetic radiation to projection optics 312 via fiber optics bundle 311. As described above with respect to FIGS. 1-3, the frequency of the electromagnetic radiation may be modified depending on the media (the area between the distal tip 310 and the target surface) the radiation is propagating through. The pattern of electromagnetic radiation may be projected onto the surface under examination by placing a slide or grating (not shown) with a desired pattern between the radiation source 303 and the fiber optics bundle 311 in the catheter section 301. The pattern of electromagnetic radiation may propagate through the fiber optics bundle 311, exit through projection optics 312 at the distal tip 310, and project onto the target surface.

The spatial radiation signals may reflect from the target surface back to the distal tip 310 and imaging optics 313 may capture the reflected signals (which are modulated by interaction with the surface). The captured reflection images may be transmitted from imaging optics 313 to camera 304 via fiber optics bundle 311 and subsequently transmitted to a controller system (not shown, but similar to controller system 106 of FIG. 1). In addition, one or more spatial sensor(s) (not shown, but similar to spatial sensor(s) 114 of FIG. 1, which may, for example, include one or more accelerometer(s), gyroscope(s), and/or IMU(s)) may gather data regarding the position and/or orientation of the distal end 302 (or a similar reference). The captured position and/or orientation information may be transmitted from the spatial sensor(s) to the controller system via fiber optics bundle 311. The controller system may use existing information regarding various signal parameters to isolate the content of the reflected spatial signal that contains the 3-D shape information. The shape information, together with the position and/or orientation data, may be used to mathematically reconstruct the 3-D shape of target surface, including identifying its location and orientation.

Moreover, endoscope 300 may be used to construct full-field surface maps of long passageways in a patient's body (e.g., gastrointestinal passageways) by moving the endoscope 300 through a given passageway. While endoscope 300 is being guided through a given passageway, continuous surface maps may be generated by stitching together the individual full-field 3-D data gathered during each video frame captured by camera 304. In addition, the full-field surface map's spatial location and orientation may be generated using the coordinate information obtained from the spatial sensor(s). The 3-D data may be stitched together using algorithms implemented in software, hardware, or a combination of software and hardware. In this manner, an accurate 3-D model of the cavity in which the device is traveling may be constantly digitally developed and recorded.

FIG. 4 illustrates a detailed, cross-sectional view of a distal end 400 that may be integrated with an endoscope described above with respect to FIGS. 3A-B according to an example embodiment of the present invention. Distal end 400 may include a lamp 401, a pattern slide 402, an illumination lens 403, an imaging sensor 404, and an imaging lens 405, electrical lead 406, data leads 407, and one or more spatial sensor(s) 408 for determining position and/or orientation (e.g., one or more accelerometer(s), gyroscope(s), and/or IMU(s)).

Lamp 401, pattern slide 402, and illumination lens 403 may form an electromagnetic radiation emitter capable of projecting patterns of radiation onto a target surface according to a spatial signal modulation algorithm. During operation, lamp 401 may receive power from a power source (not shown) via electrical lead 406 and project electromagnetic radiation through pattern slide 402 and illumination lens 403 onto a target surface.

The spatial radiation signals may reflect from the target surface back to the distal end 400 through imaging lens 405, and imaging sensor 404 may capture the reflected signals (which are modulated by interaction with the surface). Meanwhile, spatial sensor(s) 408 may gather position and/or orientation information of the distal end 400 (or similar reference). The captured reflection images and position and/or orientation information may be transmitted to a controller system (not shown, but similar to controller system 106 of FIG. 1) via data leads 407. The controller system may use information regarding various signal parameters to isolate the content of the reflected spatial signal that contains the 3-D shape information. The shape information, together with the position and/or orientation data, may be used to mathematically reconstruct the 3-D shape, location, and orientation of target surface.

FIG. 5 illustrates representative views of an endoscopic capsule 500 according to an example embodiment of the present invention. FIG. 5 includes a cross-sectional view (on the left) and an overhead view (to the right) of capsule 500. Capsule 500 may be a small vitamin pill sized capsule that is capable of being ingested by a patient. The capsule 500 may implement the SSM techniques described above to generate full-field, 3-D representations of surfaces of a human digestive tract that are difficult to reach through traditional endoscopic examination.

Capsule 500 may include one or more spatial sensor(s) 508 for collecting position and/or orientation data, imaging package 510, an electromagnetic radiation package 520, power supply and electronics 530, a wireless transmitter 540, and a transparent protective cover 550. The cover 550 may be an outer shell capable of protecting the devices in capsule 500 while it is flowing through the digestive tract of a patient. Imaging package 510 may include imaging optics 512 (e.g., a lens) and imaging sensor 514.

Capsule 500 may operate in a similar fashion to the embodiments described above, however, capsule 500 may be powered locally via power supply and electronics 530, which may include a battery, for example. Moreover, capsule 500 may transmit captured image and position/orientation data to an image processing module (not shown, but similar to controller system 106 of FIG. 1) located external to a patient's body using wireless transmitter 540. An antenna module (not shown) may be placed on the skin of the patient to facilitate data transmission from the capsule to the image processing module.

During operation, a patient may ingest capsule 500, which travels through the patient's digestive tract for measurement purposes. While capsule 500 is traveling through the patient's digestive tract, electromagnetic radiation package 520 may be powered by power supply and electronics 530 to constantly project spatial electromagnetic radiation patterns on surfaces in its path.

The spatial radiation signals may reflect from the target surface back to the imaging optics (the signals may be modulated by interaction with the surface). Image sensor 514 may capture the reflected images and transmit them, via wireless interface 540, from the capsule 500 to an image processing module (not shown, but similar to controller system 106 of FIG. 1). Meanwhile, spatial sensor(s) 508 may gather position/orientation information of the capsule 500. The image processing module may use the existing information regarding various signal parameters to isolate the content of the reflected spatial signal that contains the 3-D shape information. The shape information, together with the position and/or orientation data, may be used to mathematically reconstruct the 3-D shape, location, and orientation of the target surface.

Reflection images and position/orientation information captured by capsule 500 may be used to construct continuous surface maps within a patient's digestive tract as the capsule 500 is traveling in the tract by stitching together the individual full-field 3-D data gathered during each video frame captured by image sensor 514. In this manner, an accurate 3-D model of the cavity in which the device is traveling may be constantly digitally developed and recorded.

Embodiments of the present invention described above provide devices and methods to generate accurate, high-speed 3-D surface representations. Moreover, integrating the SSM and position/orientation techniques described above with medical devices such as probes, endoscopes, catheters, or capsules may enable physicians to generate accurate full-field, 3-D representations of surfaces that were previously very difficult to produce, while also accurately determining the location and orientation of those surfaces. There are numerous other medical applications for the techniques and devices described above.

Those skilled in the art may appreciate from the foregoing description that the present invention may be implemented in a variety of forms, and that the various embodiments may be implemented alone or in combination. Therefore, while the embodiments of the present invention have been described in connection with particular examples thereof, the true scope of the embodiments and/or methods of the present invention should not be so limited since other modifications will become apparent to the skilled practitioner upon a study of the drawings, specification, and following claims.

What is claimed is:

1. A system for full-field three-dimensional surface mapping, the system comprising:
an electromagnetic radiation source configured to project electromagnetic radiation onto a surface, the electromagnetic radiation source configured to project the electromagnetic radiation in a pattern corresponding to a spatial signal modulation algorithm and at a frequency configured for a media adjacent to the surface;
an image sensor configured to capture image data representing a reflection of the projected pattern as reflected from the surface;
one or more spatial sensors configured to capture spatial sensor data comprising one or both of position and orientation data; and
an image processing module configured to receive the captured image data and the captured spatial sensor data to calculate a full-field three-dimensional representation of the surface.

2. The system of claim 1, wherein the electromagnetic radiation source, image sensor, and spatial sensor are integrated into an endoscope.

3. The system of claim 2, wherein the image sensor and spatial sensor are electrically coupled to the image processing module.

4. The system of claim 1, wherein the electromagnetic radiation source, image sensor, and spatial sensor are integrated into a ingestible capsule.

5. The system of claim 4, wherein the image sensor and spatial sensor are wirelessly coupled to the image processing module.

6. The system of claim 1, wherein the spatial sensor is implemented using a micro-electromechanical system.

7. The system of claim 1, further comprising a display to illustrate the full-field three-dimensional representation of the surface.

8. The system of claim 1, wherein the one or more spatial sensors comprises an accelerometer.

9. The system of claim 1, wherein the one or more spatial sensors comprises a gyroscope.

10. The system of claim 1, wherein the one or more spatial sensors comprises an inertial measurement unit.

11. The system of claim 1, wherein the image processing module is configured to utilize the spatial sensor data in order to calculate the position of the surface.

12. The system of claim 1, wherein the image processing module is configured to utilize the spatial sensor data in order to calculate the orientation of the surface.

13. The system of claim 1, wherein the image processing module is configured to utilize the spatial sensor data in order to calculate the position and orientation of the surface.

14. An apparatus for full-field three-dimensional surface mapping, the apparatus comprising:
an electromagnetic radiation source configured to project electromagnetic radiation onto a surface, the electromagnetic radiation source configured to project the electromagnetic radiation in a pattern corresponding to a spatial signal modulation algorithm and at a frequency configured for a media adjacent to the surface;
an image sensor configured to capture image data representing a reflection of the projected pattern as reflected from the surface;
one or more spatial sensors configured to capture spatial sensor data comprising one or both of position and orientation data; and
an image processing module configured to receive the captured image data and the captured spatial sensor data to calculate a full-field three-dimensional representation of the surface.

15. A method for full-field three-dimensional surface mapping, the method comprising:
projecting, by electromagnetic radiation source, electromagnetic radiation onto a surface, the electromagnetic radiation being projected in a pattern corresponding to a spatial signal modulation algorithm and at a frequency configured for a media adjacent to the surface;
capturing, by an image sensor, image data representing a reflection of the projected pattern as reflected from the surface;
capturing, by one or more spatial sensors, data comprising one or both of position and orientation data; and
receiving, at an image processing module, the image data and the spatial sensor data to calculate a full-field three-dimensional representation of the surface.

16. The method of claim 15, wherein the electromagnetic radiation source, image sensor, and spatial sensor are integrated into an endoscope.

17. The apparatus of claim 16, wherein the image sensor and spatial sensor are electrically coupled to the image processing module.

18. The method of claim 15, wherein the electromagnetic radiation source, image sensor, and spatial sensor are integrated into a ingestible capsule.

19. The method of claim 18, wherein the image sensor and spatial sensor are wirelessly coupled to the image processing module.

20. The method of claim 15, wherein the spatial sensor is implemented using a micro-electromechanical system.
